(19) United States
(12) Patent Application Publication
(10) Pub. No.: US 2006/0081263 A1
(43) Pub. Date: Apr. 20, 2006

(54) Head Immobilizer

(76) Inventor: Steve Ostrowski, Hamilton, Ontario (CA)

Correspondence Address: MARK A KOCH, 866 MAIN STREET EAST, HAMILTON, ON, L8M1L9, CA

(21) Appl. No.: 11/163,331

(22) Filed: Oct. 14, 2005

Related U.S. Application Data
(60) Provisional application No. 60/618,989, filed on Oct. 18, 2004.

(51) Int. Cl. A61F 5/37

(52) U.S. Cl. 128/870

(57) ABSTRACT

The head immobilizer including a planar rack board, wherein the head immobilizer's rack board removably attached to a back board. The head immobilzer including a right wedge and a left wedge made of thin planar folded material slideably received upon the rack board for clamping a persons head between each of the wedges wherein the wedges including a locking tongue attached to the wedge and corresponding locking grooves defined in the rack board. The tongue engages with the grooves for operably locking the wedge in a preselected groove preventing the wedges from moving away from each other.

### PRIORITY CLAIM

This application claims priority from previously filed U.S. Provisional Application 60/618,989 filed on Oct. 18, 2004.

### FIELD OF THE INVENTION

The present invention relates in general to head immobilization apparatus and more specifically to head immobilization apparatus which are attachable to spine or back boards.

### BACKGROUND OF THE INVENTION

Head immobilization devices are used by emergency medical technicians to secure an injured patients head from movement, especially rotational movements of the head during transport to a medical care facility. Rigid spine and/or back boards are typically used to support the victim who is lifted or transferred onto the board. Stiff pillows or blocks are placed tightly on either side of the victims head and one or more head and/or chin straps are then tightly secured over the pillows to fixedly hold the victims head in place. The pillows or blocks can be selectively placed on the spine or backboard to accommodate different size victims heads.

Numerous devices have been proposed in the past to perform the function of immobilizing or stabilizing the human head and/or neck for emergency purposes. Such devices are extremely important, particularly in emergency situations and handling of possible fractures of the cervical spine. In the case of industrial injuries, automobile accidents and battle field injuries, it is usually necessary to remove the patient from the injury scene, often under conditions of stress or time pressure for transport to medical facilities. In handling and/or moving the patient has a high risk of aggravation of injuries to the cervical spine if the patients head or neck is not properly immobilized and stabilized for transport. The current devices in use are usually single use or disposable devices made of corrugated cardboard which do not require sterilization after use. It is desirable that such a disposable immobilizer be sturdy, but also inexpensive. Furthermore, the storage capacity of emergency medical vehicles is limited and therefore, it is important that before use the head immobilizer be as compact as possible to minimize the storage space taken up by the devices within the ambulance. It is particularly desirable that head immobilization devices can be stored in the flat condition and unfolded to an operative condition when required.

### SUMMARY OF THE INVENTION

A head immobilizer including:

(a) planar rack board including a means for removably attaching said rack board to a back board;

(b) a right wedge and a left wedge slideably received upon said rack board for clamping a person head between clamping faces of each of said wedges;

(c) a means for releasably locking said wedges in preselected positions on said rack board.

### BRIEF DESCRIPTION OF THE DRAWINGS

The present invention will now be described by way of example only with reference to the following drawings in which:

FIG. 1 is a top perspective view of the present invention a head immobilizer.

FIG. 2 is a top perspective view of the present invention a head immobilizer showing it together with the head clamped in the device.

FIG. 3 is a top perspective view of the present invention a head immobilizer shown with the wedges in closer proximity.

FIG. 4 is a cross sectional view of the head immobilizer shown in FIG. 1 with the unit in the completely folded position.

FIG. 5 is a cross sectional view of the head immobilizer with the unit in the partially unfolded position.

FIG. 6 is a cross sectional view of the head immobilizer shown in FIG. 1 in the completely unfolded position with the wedges spaced far apart.

FIG. 7 is a cross sectional view of the head immobilizer in the unfolded position with the wedges spaced closer together.

FIG. 8 is a cross sectional view present invention a head immobilizer with the wedges spaced in a even closer position.

FIG. 9 is a cross sectional view of the head immobilizer together with a back board showing the head immobilizer mounted onto a back board.

FIG. 10 is a side elevational view of the head immobilizer together with a persons head clamped into the wedges and the head immobilizer unit mounted onto a back board.

FIG. 11 is a top plan view of a die cutting pattern of one of the wedges shown in an alternate embodiment in FIG. 13, having a fold line D-118.

FIG. 12 is a top plan view of the rack board with fold line B-72.

FIG. 13 is an alternate embodiment of the head immobilizer shown generally as 120 with left and right wedges having a fold line positioned at D-118.

FIG. 14 is a top perspective view of an alternate embodiment of the present invention, a head immobilizer shown general as 120.

FIG. 15 is an upright unfolded perspective view of a wedge shown in the assembled condition, not mounted on the rack.

FIG. 16 is a top perspective view of the back portion of a wedge shown in the folded position, wherein the wedge is folded flat onto itself for shipping purposes.

FIG. 17 is a top perspective view of the wedge in a partially unassembled condition, showing how the wedge assembles to form a unfolded wedge.

### DETAILED DESCRIPTION OF THE PREFERRED EMBODIMENT

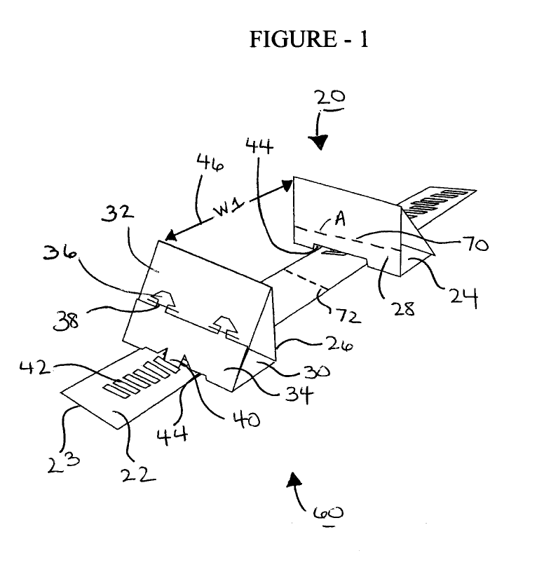

The present invention a head immobilizer shown generally as 20 in FIGS. 1 through 10, includes the following major components, namely; rack board 22, right wedge 24 and left wedge 26. Note that right wedge 24 and left wedge 26 are completely interchangeable in that the wedges are identical in construction and shape and can be used as either a left or right wedge on the rack board 22.

Right wedge 24 for example, includes the following major components, namely; clamping face 28, bottom portion 30 a back portion 32 which includes an overlap section 34 which is fastened to back portion 32 with fastening tabs 36 which pass through fastening slots 38 defined in back portion 32. A person skilled in the art will note that right wedge 24 and left wedge 26 form a triangular shape and cross section with clamping face 28 being a vertical flat plane, and wherein the bottom portion 30 and the back portion 32 serves to support and maintain the upright position of clamping face 28. Overlap section 34 of right wedge 24 or left wedge 26 includes a locking tongue 40 which cooperatively engages with locking grooves 42 defined in rack board 22.

Rack opening 44 which is defined in clamping face 28, overlap section 34 and back portion 32 allows both right wedge 24 and left wedge 26 slideably move along rack board 22. The two clamping face 28 of right wedge 24 and left wedge 26 define an opening width one (w1) shown as 46 in FIG. 1 and this opening width depends upon the relative position between right wedge 24 and left wedge 26.

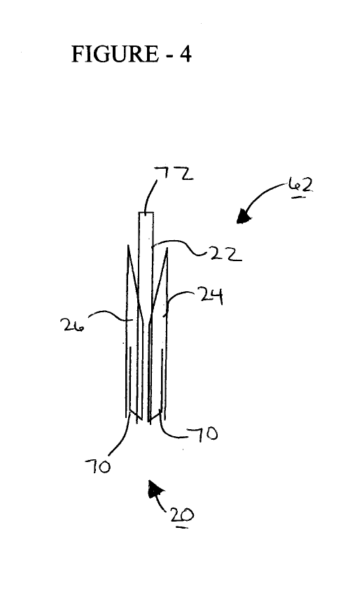

In FIG. 1, head immobilizer 20 is shown in unfolded position 60, whereas in FIG. 4 head immobilizer 20 is shown in folded position 62, wherein rack board 22 is folded along fold line B denoted as 72 in FIGS. 1 and 4 and clamping face 28 of both right wedge 24 and left wedge 26 is folded along fold line A denoted as 70 in both FIG. 1 and FIG. 4, such that rack board 22 and left wedge 26 and right wedge 24 all collapse upon each other as shown in FIG. 4.

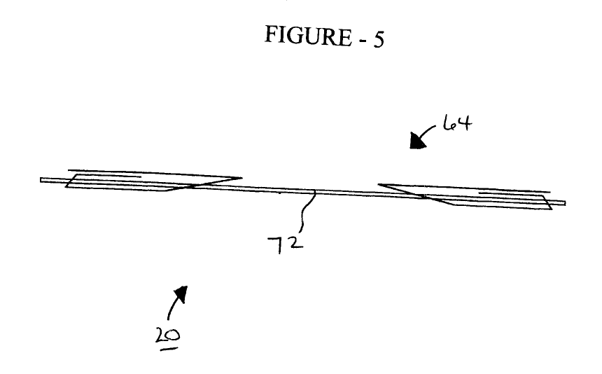

In FIG. 5, head immobilizer 20 is shown in the partially unfolded position 64 in which rack board 22 is unfolded along fold lines B shown as 72 into the position shown in FIG. 5.

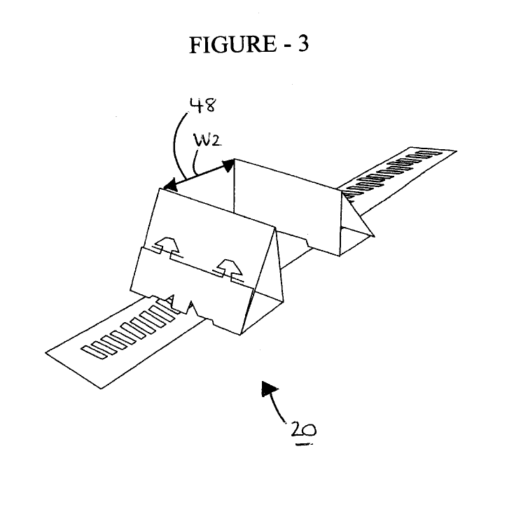

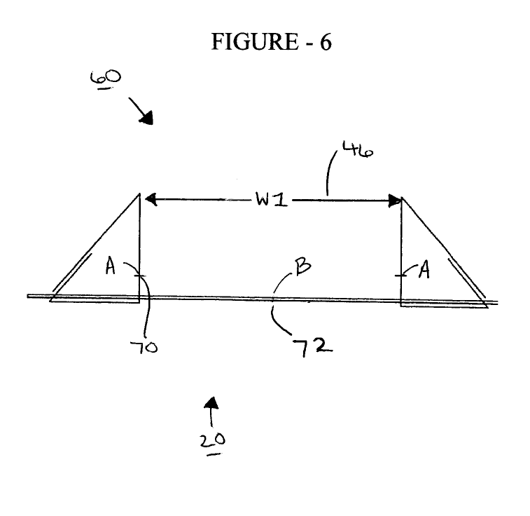

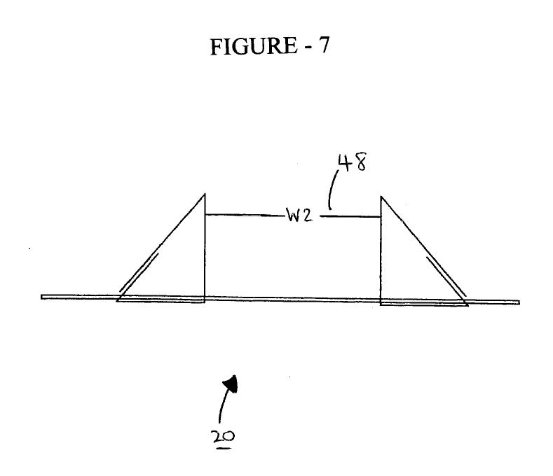

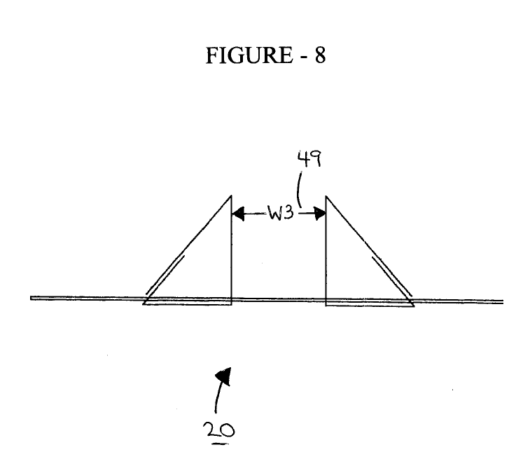

FIG. 6 shows head immobilizer 20 in the unfolded position 60 as does FIG. 1, however FIG. 6 shows head immobilizer 20 in cross sectional view indicating that the rack board 22 is unfolded along fold lines B-72 and right and left wedge 24 and 26 is unfolded along fold line A-70 as shown in FIG. 6. Both FIG. 3 and FIG. 7 show the opening at W-2 denoted as 48 and furthermore, FIG. 8 shows an opening at W-3 denoted as 49 depicting the possibility that right wedge 24 and left wedge 26 can be positioned in many different positions relative to each other.

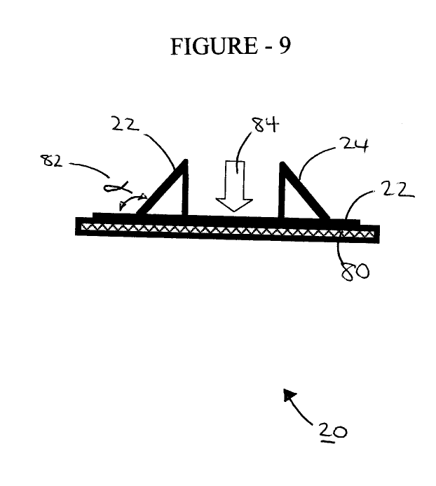

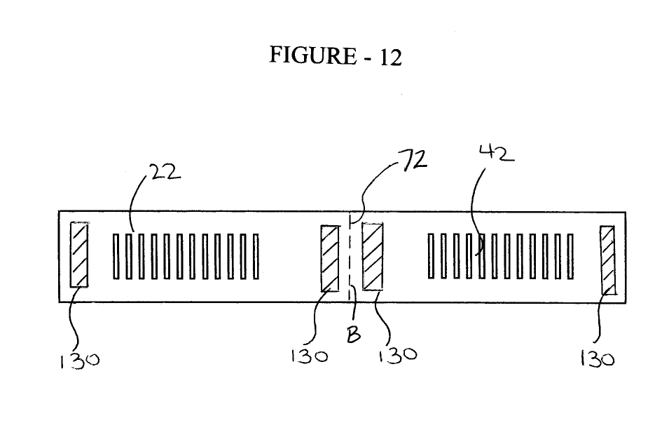

Referring now to FIG. 9, head immobilizer 20 is shown in cross-sectional view with the head immobilizer 20 mounted onto a back board 80. There are many different options for mounting rack board 22 onto back 80, however in the preferred embodiment adhesive strips shown as adhesive 130 in FIG. 12, preferrably spaced at both distal ends of rack board 22 and also centrally close to the centre line of fold line B-72 of rack board 22 is the preferred method for adhesively bonding rack board 22 onto back board 80. In this manner, removable strips can be taken off of adhesive strips 130 and then pressure placed down onto back board 80 as shown by arrow 84.

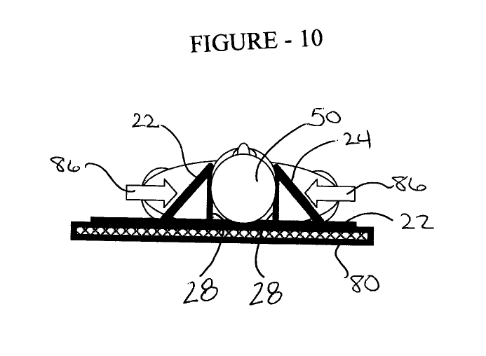

FIG. 10 shows head immobilizer 20 mounted onto a back board 80, wherein arrows 86 indicate the movement of right wedge 24 and left wedge 22 in order to immobilize or fix a persons head 50 into position, between clamping face 28.

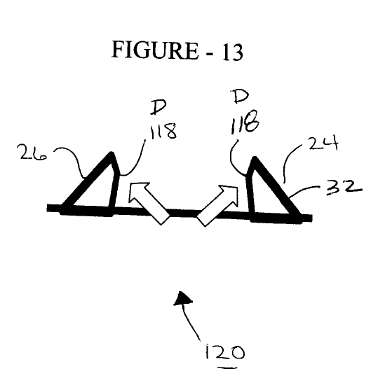

FIG. 13 shows an alternate embodiment, namely head immobilizer 120, in which clamping face 28 rather than folding at fold line “A” depicted as 70 in FIG. 6, fold at line “D” shown as 118 in FIG. 13, rather than clamping face 28 folding inwardly towards back portion 32, it folds outwardly away from back portion 32.

A person skilled in the art will note that there may be other methods of folding right and left wedge 24 and 26 onto themselves, such that they lie flat during transportation. We have shown two examples of how this can be accomplished, namely using fold lines A-70 and fold line B-72 for head immobilizer 20 or using fold lines D-118 shown in FIG. 13 and fold line B-72.

Head immobilizer 120 operates in analogous fashion as head immobilizer 20 and in all other respects functions in identical manner as head immobilizer 20 and as described below under the section in use. The only difference between head immobilizer 120 and head immobilizer 20 is the manner in which it collapses or folds into folded position 62, wherein head immobilizer 120 folds along fold line D-118 and head immobilizer 20 folds along fold lines A-70.

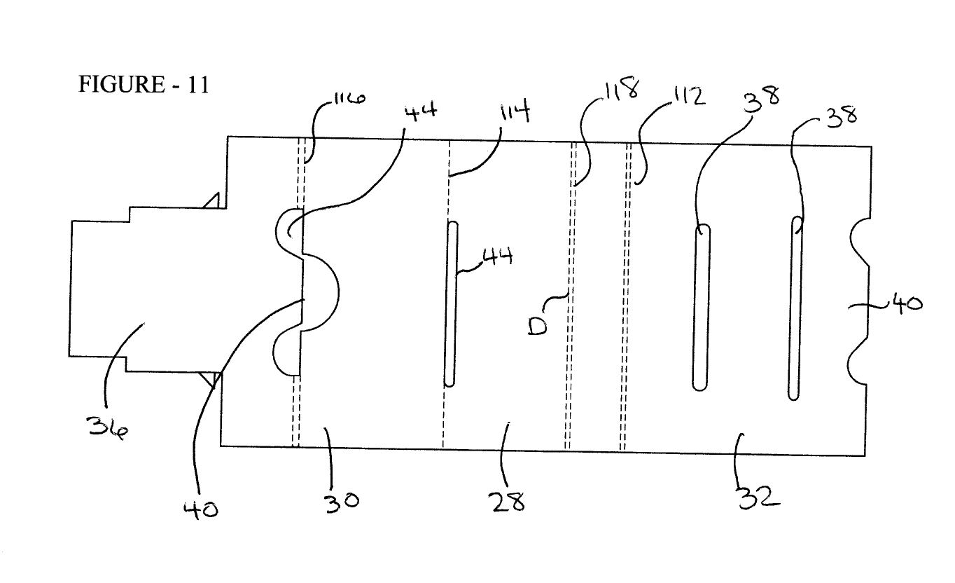

FIG. 11 shows a die cutting pattern of wedge blank 110 for either right wedge 24 or left wedge 26.

Right wedge 24 and left wedge 26 are constructed from a single piece of flat corrugated plastic and/or corrugated cardboard and is die cut and/or cut in some manner to the shape shown in FIG. 11 and scored and/or pre-bent at the fold lines and bend lines as shown in the diagram.

In particular FIG. 11 shows some of the details of right wedge 24 and left wedge 26, namely the construction of fastening tabs 36 which fit into fastening slot 38 and rack opening 44 which receives slideably there through rack board 22. FIG. 11 also shows wedge blank 110 having two tongues 40 and this is an optional feature and it is possible to manufacture or produce right wedge 24 and/or left wedge 26 with either one locking tongue 40 or two locking tongues 40.

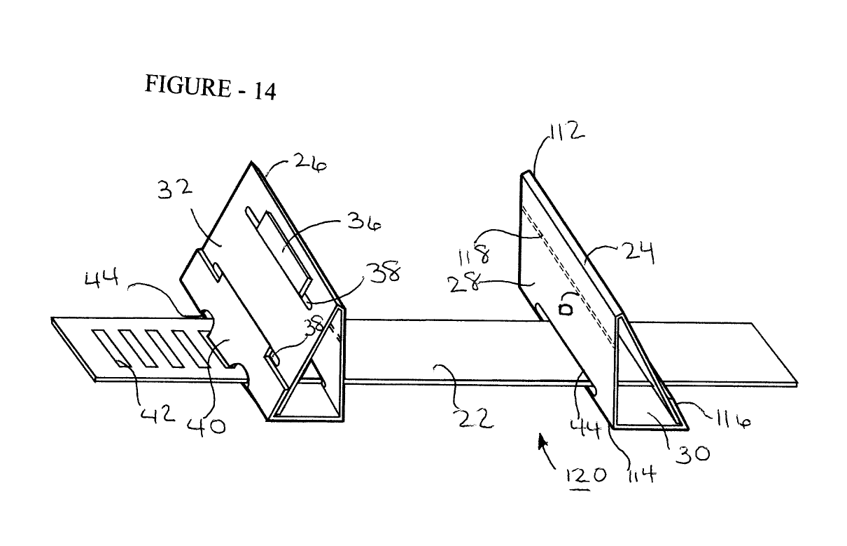

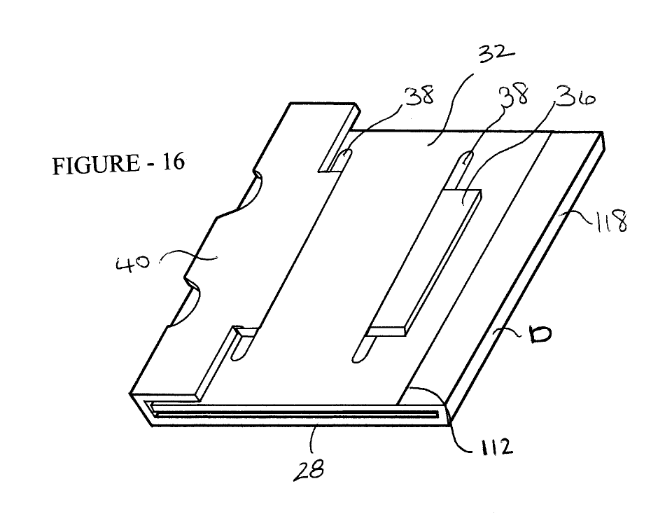

FIG. 14 shows an alternate embodiment of the present invention, head immobilizer 120 shown with right wedge 24 and left wedge 26 mounted onto rack board 22. Right and left wedges 24 and 26 respectively differ from the previous embodiment in that in the folded position the clamping face 28 of the wedge folds along fold line D-118 in contrast to fold line A-70 in the previous embodiment. In addition, in the previous embodiment, clamping face 28 of wedges 24 and 26 folded inwardly toward the back portion 32, whereas in the presently preferred embodiment as best shown and depicted in FIG. 13 as well as in FIG. 16, clamping face 28 folds outwardly along fold line D-118 away from back portion 32.

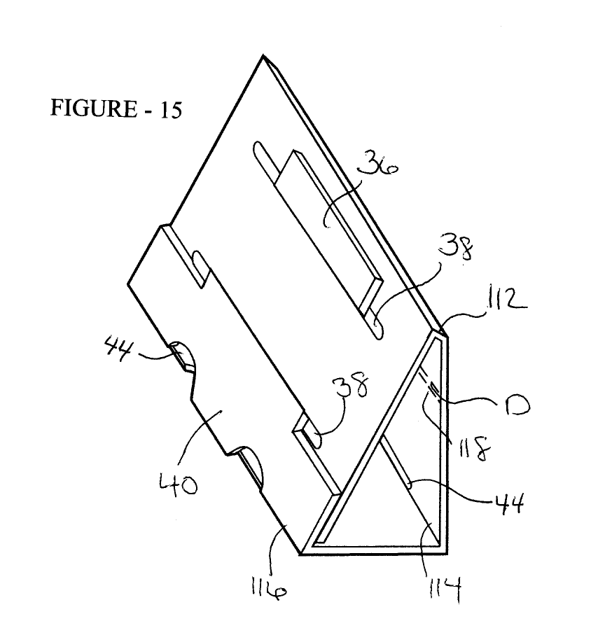

FIG. 15 shows the wedge of the presently preferred embodiment 120 in the ready to use unfolded position, whereas FIG. 16 shows the wedge of the presently preferred alternate embodiment 120 in the folded position in which clamping face 28 folds along fold line D-118, such that the clamping face 28 and the back portion 32 as well as the bottom portion 30 are folded flat onto each other.

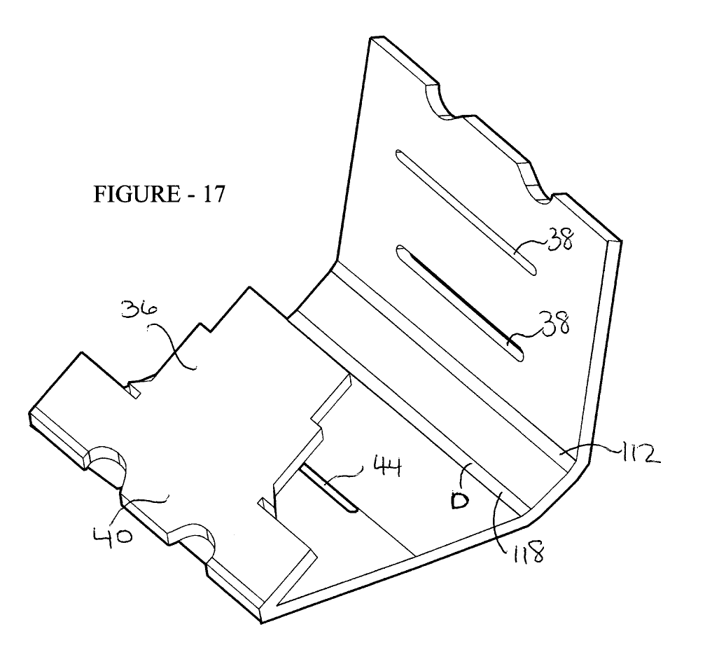

FIG. 17 shows the presently preferred alternate embodiment wedge in a partially unassembled state showing how the blank depicted in the FIG. 11 is folded and bent in order to assemble a wedge into either the unfolded position shown in FIG. 15 or the folded position shown in FIG. 16.

The advantage of the presently preferred embodiment wedge is that when pressure is placed against clamping face 28, clamping face 28 will resist folding inwardly, whereas in the previous embodiment clamping face 28 would more easily collapse or fold inwardly on itself when pressure was placed on clamping face 28.

In Use

Head immobilizer 20 is shipped in the folded position 62 as shown in FIG. 4 and upon receipt, the user would unfold head immobilizer 20, by firstly unfolding it along fold line B depicted as 72, such that head immobilizer 20 is then unfolded to partially unfolded position 64 shown in FIG. 5 and then furthermore, right and left wedge 24 and 26 are unfolded along fold lines A denoted as 70, such that both right and left wedge 24 and 26 are brought to the unfolded position shown as 60 in FIG. 6. In unfolded position 60, right wedge 24 and left wedge 26 may be at some arbitrary position along rack board 22 as shown in FIG. 1 for example and right wedge 24 and left wedge 26 can be slideably moved along rack board 22 by either slideably urging right wedge 24 and/or left wedge 26 closer to the centre of rack board 22, wherein locking tongue 40 automatically disengages from the locking groove 42 moves into the next locking groove 42 that it meets up with. In this manner opening width W-1, shown as 46 can become narrow by forcibly urging right wedge 24 and left wedge 26 together.

It is also possible to move right wedge 24 and left wedge 26 away from each other by lifting locking tongue 40 out of locking groove 42 by placing ones fingers into finger opening 90 and thereby manually lifting locking tongue 40 out of locking groove 42. Once locking tongue 40 is clear of locking groove 42, left wedge 26 or right wedge 24 can be moved away from each other or away from the centre of rack board 22 therefore creating a greater opening W-1, denoted as 46 in FIG. 1.

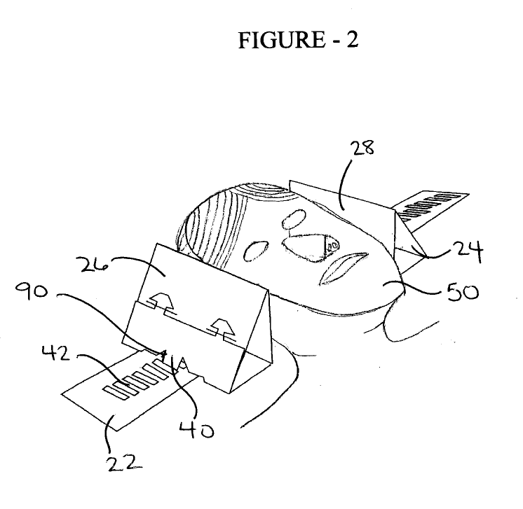

The purpose of head immobilizer 20 is to clamp a head between clamping face 28 of right wedge 24 and left wedge 26 as shown in FIGS. 2 and 10. This may be a requirement, when for example an injured person has suffered neck trauma and thereby the paramedics wish to immobilize the neck and head portion of a person when they are placed on a back board. Rack board 22 further includes a bottom surface 23 which may have an adhesive applied thereto such that rack board 22 can be adhesively affixed to a back board upon which the patient is securely strapped.

Head immobilizer 20 provides for two vertical planer surface namely clamping face 28 which are found on both right wedge 24 and left wedge 26 and require no assembly, other than unfolding the head immobilizer along fold line A-70 and fold line B-72 prior to use.

A positive lock is provided by locking tongue 40 engaging with locking grooves 42 and by providing for a large number of locking grooves 42, one can provide for a very fine adjustment in opening W-1 shown as 46.

In the preferred embodiment of this invention, the components are made out of corrugated poly propylene of a pre-selected thickness. It may also be possible to make the unit out of cardboard and/or other sheet materials having suitable strength properties.

Locking tongue 40 is locked into locking groove 42 simply by the geometry created between right and left wedge 24 and 26 and rack board 22. Locking tongue 40 is dimensioned to be slightly longer and projecting into the rack board 22 such that right wedge and/or left wedge 24 and 26 are slideably moved along rack board 22, locking tongue 40 automatically projects into locking grooves 42. In order to retract or open up the gap between right and left wedge 24 and 26, one must manually use fingers in finger openings 90 to manually lift locking tongue 40 out of locking groove 42, thereby allowing right and left wedge 24 and 26 to be moved slideably further apart from each other.

It should be apparent to persons skilled in the arts that various modifications and adaptation of this structure described above are possible without departure from the spirit of the invention the scope of which defined in the appended claim.

What is claimed is:

1. A head immobilizer including a planar rack board including a means for removably attaching said rack board to a back board, a right wedge and a left wedge slideably received upon said rack board for clamping a person head between each of said wedges and a means for releasably locking said wedges in preselected positions on said rack board.

2. The head immobilizer claimed in claim 1 wherein the wedges are fabricated from a single blank of flat planar material which is folded into a three sided triangular shape.

3. The head immobilizer claimed in claim 2 wherein the one continuous planar blank including the following integral sections connected together at bend lines a back portion connected at one end to a clamping face, which is connected at the other end to a bottom portion such that in an unfolded position the blank forms the wedge which is ready for use.

4. The head immobilizer claimed in claim 3 wherein the wedges include rack opening slots defined within back portion and clamping face for slideably receiving rack board there through.

5. The head immobilizer claimed in claim 1 wherein the releasably locking means operably permitting the wedges to move towards each other unrestricted.

6. The head immobilizer claimed in claim 4 wherein the releasably locking means including a locking tongue attached to the wedge and corresponding locking grooves defined in said rack board wherein the tongue engages with the grooves for operably locking the wedge in a preselected groove preventing the wedges from moving away from each other.

7. The head immobilizer claimed in claim 1 wherein the rack board together with the wedges mounted thereon fold together into a folded position to form a layered substantially flat unit.

8. The head immobilizer claimed in claim 1 wherein the wedges fold from a folded position to an unfolded position in which the head immobilizer is ready for use.

9. The head immobilizer claimed in claim 3 wherein the clamping face including a fold line D laterally across the clamping face, such that the clamping face folds at fold line D for operably folding the wedge between a folded position to an unfolded position.

10. The head immobilizer claimed in claim 8 wherein the fold line D permits the clamping face to fold away from the back portion when collapsing the wedge from the unfolded to folded position.

11. The head immobilizer claimed in claim 8 wherein in the folded position the planar faces of the clamping face, back portion and bottom portion are folded such that they lie flat and adjacent each other.

12. The head immobilizer claimed in claim 1 wherein the head immobilzer is manufactured from flat planar corrugated plastic.

13. The head immobilizer claimed in claim 2 wherein the flat planar material includes corrugated plastic.

14. The head immobilizer claimed in claim 3 wherein the wedge further including an overlapped section connected at one end to the bottom portion at a bend line, and at the other end including a fastening tab projecting there from such that in the unfolded position overlapped section overlaps with the back portion and fastening tab engages with fastening slots defined in the back portion thereby preventing disengagement of the back portion from the bottom portion.

15. The head immobilizer claimed in claim 3 wherein the wedge in the ready for use unfolded position the clamping face is disposed substantially vertically upright, the bottom portion is disposed substantially horizontally and the back portion is disposed slanted on an angle such that the horizontal bottom portion and slanted back portion serve to maintain the upright vertical position of the clamping face.

16. The head immobilizer claimed in claim 4 wherein the releasably locking means can be manually disengaged using finger pressure to remove locking tongue from engagement with the locking grooves thereby permitting wedge to slidably move freely along back board.
